(19) United States
(12) Patent Application Publication
(10) Pub. No.: US 2020/0066396 A1
(43) Pub. Date: Feb. 27, 2020

(54) SIMILAR IMAGE DISPLAY CONTROL APPARATUS, SIMILAR IMAGE DISPLAY CONTROL SYSTEM, SIMILAR IMAGE DISPLAY CONTROL METHOD, DISPLAY CONTROL APPARATUS, DISPLAY CONTROL SYSTEM, DISPLAY CONTROL METHOD, AND RECORDING MEDIUM

(71) Applicant: CASIO COMPUTER CO., LTD., Tokyo (JP)

(72) Inventors: Akane MINAGAWA, Matsumoto-shi (JP); Hiroshi KOGA, Matsumoto-shi (JP); Kazuhisa MATSUNAGA, Tokyo (JP); Akira HAMADA, Sagamihara-shi (JP)

(21) Appl. No.: 16/550,899

(22) Filed: Aug. 26, 2019

(30) Foreign Application Priority Data
Aug. 27, 2018 (JP) 2018-158048
Jul. 1, 2019 (JP) 2019-122644

(51) Int. Cl. G16H 30/40; G06T 7/00; G06K 9/62

(52) U.S. Cl. CPC G16H 30/40; G06K 9/6267; G06T 7/0012

(57) ABSTRACT

A similar image display control apparatus includes a processor configured to
acquire similar images obtained as a result of a similar image search with respect to a query image,
set categories into which the acquired similar images are to be classified,
determine, in a space having a prescribed number of dimensions that is no less than two, coordinates indicating a position of each category region in accordance with attributes of types equal in number to the number of dimensions, the category region being a region indicating one of the set categories,
classify the acquired similar images into the set categories, and
place the similar images, classified into each of the categories, within the category region positioned in a position as indicated by the determined coordinates and cause a display to display the placed similar images.

### CROSS-REFERENCE TO RELATED APPLICATION

This application claims the benefit of Japanese Patent Application No. 2018-158048, filed on Aug. 27, 2018 and Japanese Patent Application No. 2019-122644 filed on Jul. 1, 2019, the entire disclosures of which are incorporated by reference herein.

### FIELD

The present disclosure relates generally to a similar image display control apparatus, a similar-image display control system, a similar image display control method, a display control apparatus, a display control system, a display control method, and a recording medium.

### BACKGROUND

In dermatology, diagnosing skin disease is a very difficult task that requires expertise. Recently, techniques are being developed for image-capturing a disease-affected area and analyzing the captured image with use of a computer. Such techniques involve compiling a database of a large volume of disease cases, performing a similar image search using a captured image of a disease-affected area of a patient as a query image, and then diagnosing the disease-affected area of the patient based on similar disease cases.

As an example of an apparatus that displays similar images, for example, Unexamined Japanese Patent Application Kokai Publication No. 2010-250529 describes an image searching apparatus and the like that extracts similar images that are similar to a query image from a database of registered images, arranges the extracted similar images on the periphery of the query result, and presents, to display means, a search result in which the query image and the similar images are connectedly displayed.

In order to support the diagnosis, techniques for determining whether a disease-affected area is benign or malignant are also being developed. For example, in “Nevisense—a breakthrough in non-invasive detection of melanoma”, [online], [Searched Jun. 14, 2019] on the Internet (URL: https://scibase.com/the-nevisense-product/), a diagnostic support apparatus that visually provides a benign/malignant skin disease ratio using single-axis information is described.

### SUMMARY

A similar image display control apparatus of the present disclosure includes a processor configured to

acquire similar images obtained as a result of a similar image search with respect to a query image,

set categories into which the acquired similar images are to be classified,

determine, in a space having a prescribed number of dimensions that is no less than two, coordinates indicating a position of each category region in accordance with attributes of types equal in number to the number of dimensions, the category region being a region indicating one of the set categories,

classify the acquired similar images into the set categories, and

place the similar images, classified into each of the categories, within the category region positioned in a position as indicated by the determined coordinates and cause a display to display the placed similar images.

Also, a display control apparatus of the present disclosure includes a processor configured to

acquire a malignant index representing a possibility that an attribute of a disease of a diagnosis target area is malignant and a first disease attribute index representing a possibility that an attribute of the disease of the diagnosis target area is a prescribed first disease attribute, and

cause the acquired malignant index and the acquired first disease attribute index to be displayed in association with each other on a display.

### BRIEF DESCRIPTION OF THE DRAWINGS

A more complete understanding of this application can be obtained when the following detailed description is considered in conjunction with the following drawings, in which:

FIG. 1 is a diagram illustrating a functional configuration of a similar image display apparatus according to Embodiment 1 of the present disclosure;

FIG. 2 is a diagram illustrating an example of category positions determined by a position determiner according to Embodiment 1;

FIG. 3 is a diagram illustrating an example of a similar image display by an image display controller according to Embodiment 1;

FIG. 4 is a flowchart of similar image display processing of the similar image display apparatus according to Embodiment 1;

FIG. 5 is a diagram illustrating an example of a comparison display screen according to Embodiment 1;

FIG. 6 is a diagram illustrating an example of a similar image display by an image display controller according to a first modified example of the present disclosure;

FIG. 7 is a diagram illustrating another example of a similar image display by an image display controller according to a second modified example of the present disclosure;

FIG. 8 is a diagram illustrating a functional configuration of a display control apparatus according to Embodiment 2 of the present disclosure;

FIG. 9 is a diagram illustrating an example of a display by the display control apparatus according to Embodiment 2;

FIG. 10 is a flowchart of display control processing of the display control apparatus according to Embodiment 2;

FIG. 11 is a flowchart of risk boundary line generation processing of the display control apparatus according to Embodiment 2;

FIG. 12 is a diagram illustrating a functional configuration of a display control apparatus according to Embodiment 3 of the present disclosure;

FIG. 13 is a diagram illustrating an example of a display by the display control apparatus according to Embodiment 3;

FIG. 14 is a flowchart of display control processing of the display control apparatus according to Embodiment 3 of the present disclosure;

FIG. 15 is a diagram illustrating a functional configuration of a display control apparatus according to Embodiment 4 of the present disclosure;

FIG. 16 is a diagram illustrating an example of a display by the display control apparatus according to Embodiment 4; and

FIG. 17 is a flowchart of display control processing of the display control apparatus according to Embodiment 4.

### DETAILED DESCRIPTION

A similar image display apparatus and the like according to embodiments of the present disclosure are described below with reference to the accompanying drawings. Throughout the drawings, components that are the same or equivalent are assigned the same reference signs.

### Embodiment 1

A similar image display apparatus 100 according to Embodiment 1 of the present disclosure collects, for each prescribed category, search images obtained as a result of a similar image search with respect to a query image, and arranges the search images within the categories based on the degree of similarity with the query image. A relationship between similar images can be displayed in a manner that is easy to understand by arranging and displaying, in an n-dimensional space defined by a prescribed axis or axes, categories into which the similar images are collected and arranged. The manner in which such a display is performed is described below.

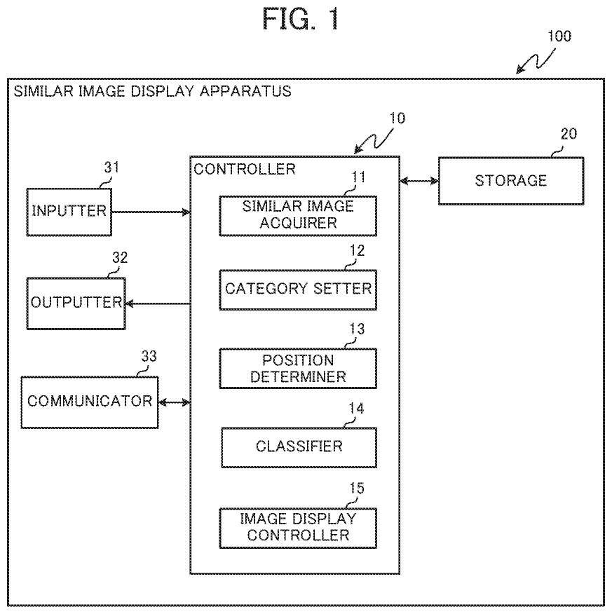

The similar image display apparatus 100 according to Embodiment 1, as illustrated in FIG. 1, includes a controller 10, a storage 20, an inputter 31, an outputter 32, and a communicator 33.

The controller 10 includes, for example a central processing unit (CPU), and executes programs stored in the storage 20 to achieve the functions of individual components (similar image acquirer 11, category setter 12, position determiner 13, classifier 14, and image display controller 15), which are described further below.

The storage 20 includes a read-only memory (ROM), a random access memory (RAM), and the like, and stores programs to be executed by the CPU of the controller 10 and necessary data.

The inputter 31 is a device used by a user of the similar image display apparatus 100 to input instructions directed at the similar image display apparatus 100 and input query images. Examples of the inputter 31 include a keyboard, mouse, touch panel, camera, and the like. The controller 10 acquires instructions and query images from the user via the inputter 31. Any device can be used as the inputter 31 as long as the controller 10 can acquire instructions or query images from the user. Moreover, the controller 10 may acquire query images via the communicator 33. The term query image refers to image data to be inputted when conducting a search for similar images that are to be displayed on the similar image display apparatus 100. The similar image display apparatus 100 presents, to the user, images that are similar to the query image in an easy to understand manner.

The outputter 32 is a device used by the controller 10 to present similar images to the user. Examples of such devices include a display, an interface for a display, and the like. The similar image display apparatus 100 may include the outputter 32 as a display, and may display a search result or the like on an external display connected via the outputter 32. The similar image display apparatus 100 without the display (similar image display apparatus 100 in which the outputter 32 is an interface for the display) is also referred to as the similar image display control apparatus.

The communicator 33 is a device (network interface, for example) for transmitting and receiving data to and from another external device (server storing a database of image data, or a similar image searching device, for example). The controller 10 can acquire query images and images similar to the query image via the communicator 33.

Next, the function of the controller 10 is described. The controller 10 achieves the functions of a similar image acquirer 11, the category setter 12, the position determiner 13, the classifier 14, and the image display controller 15.

The similar image acquirer 11 acquires data (image data of similar images and a degree of similarity between these images and the image query) obtained as a result of the similar image search with respect to the query image. Specifically, the similar image acquirer 11 acquires data of images that have a degree of similarity that is greater than or equal to a prescribed threshold in the similar image search and also acquires the degree of similarity. The similar image acquirer 11 may acquire data of similar images obtained as a result of the search by the controller 10 for images that are similar to the query image, and for example, may cause an external similar image searching device to search, via the communicator 33, for images that are similar to the query image, and may also acquire data of the similar images searched by the similar image searching device. Also, the image data is appended with their own corresponding information such as the disease names associated in one-to-one correspondence to the images as tag information.

The category setter 12 sets a category group (plurality of categories) into which images acquired by the similar image acquirer 11 are classified. In a case where the target is image data of skin, this category group is, “disease name” (pigmented nevus, melanoma, basal cell carcinoma, or the like), “outer shape” (round, star-shaped, elliptical, or the like), “color” (red, black, brown, or the like), “size”, “internal structure”, “nevus (pigmented spot) state” (mesh pattern, globular pattern, cobblestone pattern, homogenous pattern, parallel pattern, starburst pattern, multi-component pattern, unspecific pattern) or the like. For example, in a case in which the category group is the disease name, one category for each of the specific disease names: pigmented nevus, melanoma, and basal cell carcinoma is created. Information of the category group (plurality of categories) into which images are classified is stored in advance in the storage 20. The category setter 12 sets the category group (plurality of categories) into which image data is classified, based on the information of the category group stored in the storage 20.

The position determiner 13 determines a position where a region indicating each category included in the category group (plurality of categories) set by the category setter 12 is displayed as coordinates in an n-dimensional space based on n-types of attributes (n being an integer greater than or equal to one). More specifically, each attribute of n-types of attributes is associated in one-to-one correspondence with a coordinate axis of n-axes defining the coordinates of the n-dimensional space, and the coordinates indicating the position where the individual regions (category region), each indicating a category, is to be displayed is based on attribute values of individual attributes, each individual attribute corresponding to a particular coordinate axis of the coordinate axes.

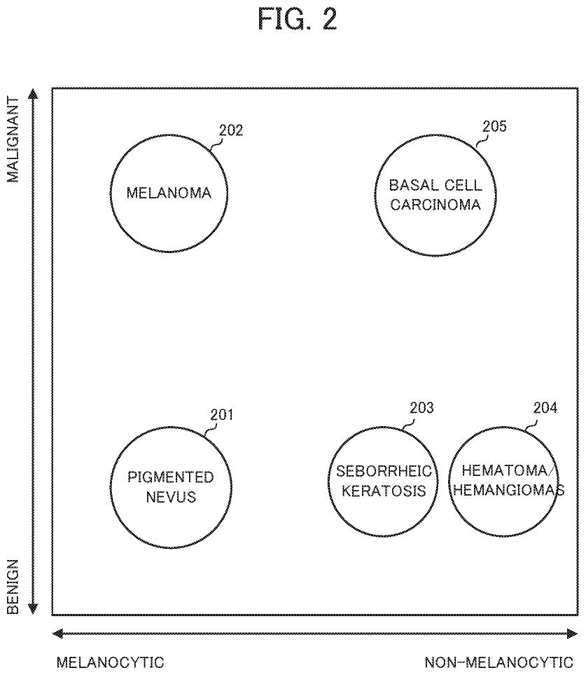

A case in which the disease name is set as the category group by the category setter 12 and the position determiner 13 determines positions within a two-dimensional space of the category group (disease name) with two types of attributes “benign/malignant” and “melanocytic/non-melanocytic” is considered as an example. In this case, the position determiner 13, for example as illustrated in FIG. 2, determines the coordinates of the positions where the disease names are displayed in the two-dimensional space with “benign/malignant” being placed on the vertical axis (Y-axis) and “melanocytic/non-melanocytic” being placed on the horizontal axis (X-axis). Here, on the vertical axis (Y-axis), malignant is placed on the upper side whereas benign is placed on the lower side, and on the horizontal axis (X-axis), melanocytic is placed on the left side whereas non-melanocytic is placed on the right side.

If, as a specific example, the following five disease names: pigmented nevus, melanoma, seborrheic keratosis, hematoma/hemangiomas, and basal cell carcinoma are considered, the attributes for the diseases are as follows: “benign, melanocytic” for pigmented nevus, “malignant, melanocytic” for melanoma, “benign, non-melanocytic” for seborrheic keratosis, “benign, non-melanocytic” for hematoma/hemangiomas, and “malignant, non-melanocytic” for basal cell carcinoma. Therefore, the position determiner 13, as illustrated in FIG. 2, determines the positions for each of the diseases as follows: the lower left region for pigmented nevus 201, the upper left region for melanoma 202, the lower right region (a little to the left of the region center) for seborrheic keratosis 203, the lower right region (a little to the right of the region center) also for hematoma/hemangiomas 204, and the upper right region for basal cell carcinoma 205.

The position determiner 13 may adjust the display positions of the categories as necessary so that the positions where different categories are displayed have different coordinates. For example, in the example illustrated in FIG. 2, since seborrheic keratosis 203 and hematoma/hemangiomas 204 are both “benign, non-melanocytic”, both categories will be displayed in the same bottom right region unless the display positions are adjusted. Therefore, in the example illustrated in FIG. 2, the position determiner 13 adjusts the display positions such that both diseases are displayed. Specifically, the position determiner 13 places seborrheic keratosis 203 in a shifted position being slightly to the left of the center of the bottom right region, and places hematoma/hemangiomas 204 in a shifted position being slightly to the right of the center of the bottom right region.

Information of the n-types of attributes that is used for determining the coordinate axes in a space, information of the attributes for each of the categories, and placement information for the individual attributes, for the position determiner 13 to determine the display positions for each of the categories, is stored in advance in the storage 20. The position determiner 13 determines the coordinates, in the n-dimensional space, of the positions where the category group (plurality of categories) is displayed, based on the information of the n-types of attributes, information of the attributes for each of the categories, and placement information for the individual attributes. In the example illustrated in FIG. 2, the information of two types of attributes, namely, the “benign/malignant” attribute and the “melanocytic/non-melanocytic” attribute, are stored in the storage 20 as attribute information. Also, the following information: pigmented nevus 201 is “benign, melanocytic”, melanoma 202 is “malignant, melanocytic”, seborrheic keratosis 203 is “benign, non-melanocytic”, hematoma/hemangiomas 204 is “benign, non-melanocytic”, and basal cell carcinoma 205 is “malignant, non-melanocytic” is stored in the storage 20 as information of the attributes for each category. Also, the following information: “benign” of the “benign/malignant” attribute is placed on the lower side whereas “malignant” of the “benign/malignant” attribute is placed on the upper side and “melanocytic” of the “melanocytic/non-melanocytic” attribute is placed on the left side whereas “non-melanocytic” of the “melanocytic/non-melanocytic” attribute is placed on the right side is stored in the storage 20 as placement information for each of the attributes.

The classifier 14 classifies image data acquired by the similar image acquirer 11 into one of the categories of the category group (plurality of categories) set by the category setter 12. The classifier 14 can classify image data by use of tag information that is appended to the image data (for example, the disease name is appended as tag information to each image).

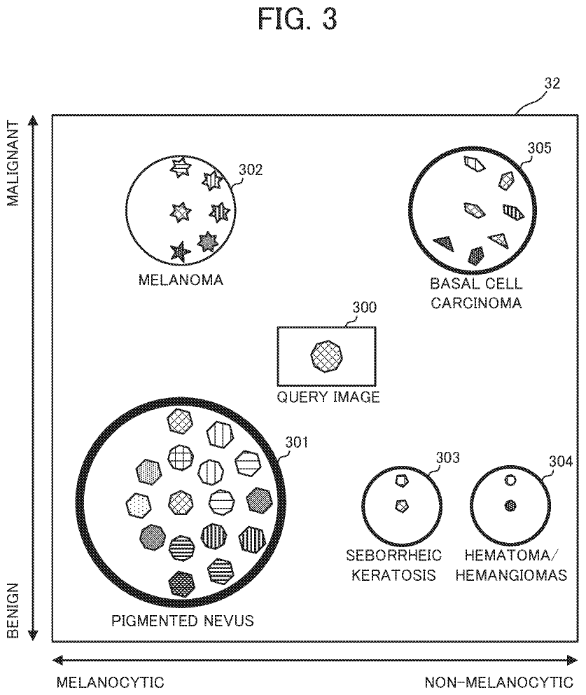

The image display controller 15 places, based on the degree of similarity with the query image, the image data, which is classified into each of the categories by the classifier 14, inside the regions of the respective categories whose coordinates were determined in the n-dimensional space by the position determiner 13. The image display controller 15 displays the image data accordingly via the outputter 32. The image display controller 15, for example as illustrated in FIG. 3, places the similar images classified into pigmented nevus into a pigmented nevus region 301 (within circle in bottom left portion of FIG. 3), the similar images classified into melanoma into a melanoma region 302 (within the circle in the upper left portion of FIG. 3), the similar images classified into seborrheic keratosis into a seborrheic keratosis region 303 (within the circle that is slightly to the left in the bottom right portion of FIG. 3), the similar images classified into hematoma/hemangiomas into a hematoma/hemangiomas region 304 (within the circle that is slightly to the right in the bottom right portion of FIG. 3), and the similar images classified into basal cell carcinoma into a basal cell carcinoma region 305 (within the circle in the upper right portion of FIG. 3), and, the greater the degree of similarity these classified similarity images have with a query image 300, the closer to the center of their respective region (within the circle) they are placed and displayed.

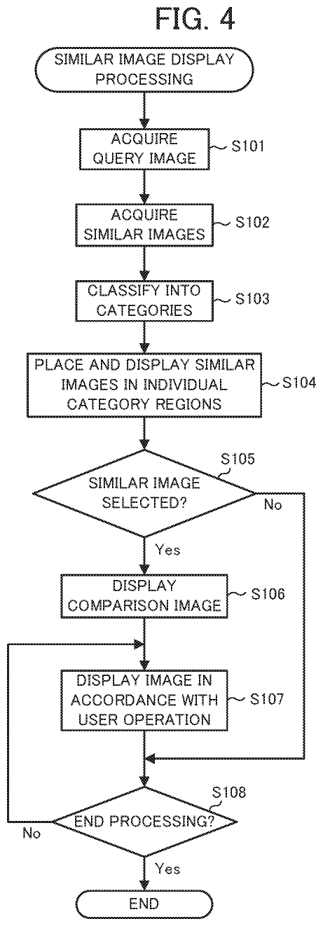

The functional configuration of the similar image display apparatus 100 is described above. Details of the similar image display processing performed by the similar image display apparatus 100 are described next with reference to FIG. 4. The similar image display processing begins when the user instructs the similar image display apparatus 100, via the inputter 31, to start the similar image display processing.

First, the controller 10 of the similar image display apparatus 100 acquires a query image (step S101). For example, when the user inputs the query image into the similar image display apparatus 100 via the inputter 31 (drags and drops the query image into a prescribed region on the screen, for example), the controller 10 acquires the query image.

Next, the similar image acquirer 11 acquires similar images obtained as a result of a similar image search with respect to the query image (step S102). Specifically, similar images that have a degree of similarity with the query image that are greater than or equal with a prescribed threshold are acquired. At such time, the similar image acquirer 11 acquires the similar image together with the degree of similarity that the similar image has with the query image. Step S102 is also referred to as the similar image acquisition step. The processing of the similar image search may be performed by an external similar image searching device instead of the similar image display apparatus 100. In such a case, the controller 10 transmits the query image acquired in step S101 to the external similar image searching device via the communicator 33 and the similar image acquirer 11 acquires the result of the similar image search performed by the external similar image searching device.

Then, the classifier 14 classifies the similar images acquired by the similar image acquirer 11 into categories set by the category setter 12, based on tag information appended to each similar image (step S103). Step S103 is also referred to as the classification step.

Next, the image display controller 15 places the similar images, which are classified into each of the categories in step S103, in the regions of the categories whose positions were determined by the position determiner 13 and displays these similar images via the outputter 32 (step S104). Specifically, as illustrated in FIG. 3, in each the region of each category, the greater the degree of similarity with the query image, the closer toward the center of the region of the category the image is placed in a concentric circular manner. In the example illustrated in FIG. 3, the image having the greatest degree of similarity with the query image, among the similar images classified into a particular category, is placed in the center of the particular category. The image having the second greatest degree of similarity is placed above the center and other images are placed clockwise thereafter in descending order of degree of similarity in a concentric circular manner.

Also, in step S104, the image display controller 15 displays, in the region of each category, a circle of a size in accordance with the number of similar images that are classified into the particular category. The displaying of these circles makes it easy to intuitively grasp the scale of each category. Also, the greater the degree of similarity is between an image at the center (the similar image having the greatest degree of similarity with the query image for a particular category) and the query image, the thicker the width of the circumferential line of the circle is displayed by image display controller 15. The thickening of the width of the circumferential line of the circle in such a manner makes it easy for the user to intuitively grasp placement location of similar images that are most similar to the query image. Also, thickness of the circumferential line of this circle does not need to be displayed at a prescribed thickness that is in accordance with the degree of similarity between the image at the center of the circle and the query image. The image display controller 15, for example, may display a prescribed thickness of the circumferential line of the circle in accordance with a degree of similarity between a prescribed image and the query image. Here the prescribed image is, for example, an image in a particular category having an n-th (n being an integer that is no less than 1 and no greater than the number of similar images classified in that particular category) greatest degree of similarity with the query image, a lowest degree of similarity, or a middlemost image when arranged in order of degree of similarity. Also, displaying at the prescribed thickness means, for example, that the thickness is displayed thicker the greater the degree of similarity, and displayed thinner the lower the degree of similarity. In order to ensure that the user can easily compare each similar image with the query image, the image display controller 15, in step S104, also performs processing for displaying the query image 300 in the center portion of the display screen as illustrated in FIG. 3. The step S104 is also referred to as the image display control step.

Next, the controller 10 determines whether or not a similar image displayed in step S104 is selected (clicked by the user, for example) via the inputter 31 (step S105). If no similar image is selected (NO in step S105), processing advances to step S108.

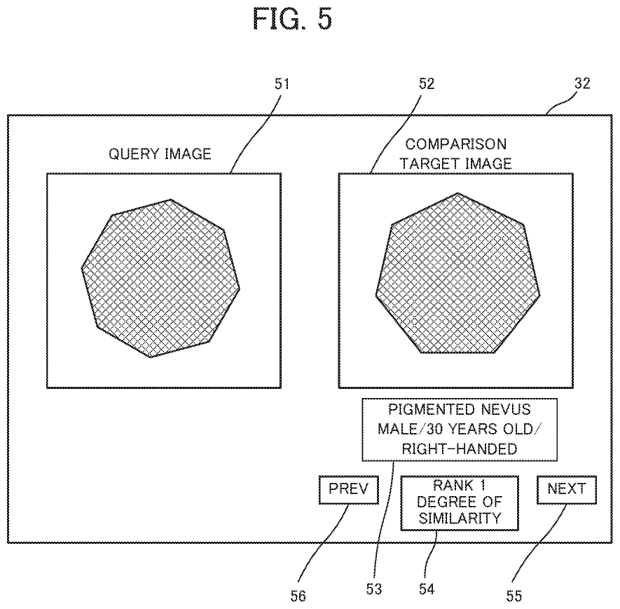

If a similar image is selected (YES in step S105), the image display controller 15 displays the image selected in step S105 and the query image in an enlarged manner so that these images can be compared (step S106). For example, in a case in which the image (image that is most similar to the query image among the similar images that are classified into pigmented nevus) at the center of the pigmented nevus in FIG. 3 is selected as a comparison target image, a comparison screen displaying a query image 51 and a clicked comparison target image 52 in an enlarged manner is displayed via the outputter 32 as illustrated in FIG. 5. In FIG. 5, the image display controller 15 also indicates, beneath the comparison target image 52, tag information 53 that is appended to the comparison target image 52, a rank 54 of a degree of similarity between the comparison target image 52 and the query image 51, a NEXT button 55 and a PREV button 56 for switching in the order of rank of similarity to another comparison target 52 having another degree of similarity.

Then, the image display controller 15 displays an image in accordance with a user operation (Step S107). For example, when a drag operation is performed on the query image 51 or the comparison target image 52, the image display controller 15 moves the image parallel to the dragging direction. When a mouse wheel rotation is performed on the query image 51 or the comparison target image 52, the image display controller 15 enlarges or reduces the size the image of the image. When the query image 51 is doubled-clicked, the image display controller 15 displays the query image display screen illustrated in FIG. 3 again. Also, when the PREV button 55 or the NEXT button 56 is clicked, the image display controller 15 can switch to another comparison target 52 having a degree of similarity of a different rank.

Next, the controller 10 determines whether or not an instruction was given to end the similar image display processing (step S108). If no instruction was given to end the similar image display processing (NO in step S108), the controller 10 returns processing to step S107. If an instruction is given to end the similar image display processing (YES in step S108), the similar image display processing is ended. For example, if an instruction to end the similar image display processing is given by the user via the inputter 31, the similar image display processing is ended.

As described above, since the similar image display apparatus 100 can classify images into categories and then place and display the similar images, for each category, in descending order of degree of similarity with the query image, the similar image display apparatus 100 can display the relationship between similar images in a manner that is easier to understand.

For example, in a case in which images of skin diseases are to be displayed, although melanoma, basal cell carcinoma, and solar keratosis are all malignant diseases, the degree of malignancy (the effect on the human body) greatly differs depending on the skin disease. Therefore, the malignancy information (attribute values of attributes) such as “malignancy 10, melanocytic” for melanoma, “malignancy 8, non-melanocytic” for basal cell carcinoma, and “malignancy 3, non-melanocytic” for solar keratosis are stored in the storage 20 as information of attributes for the individual categories, and upon determination by the position determiner 13 of the position of the categories according to malignancy such that, for example, the categories of greater malignancy are displayed as categories in circles towards the top portion of the screen, the user can confirm both the similar images placed in the individual categories and the malignancy of the individual categories. Also by determining the position of other attributes based likewise on attribute values of the other attributes, the user is able to confirm the similar images in accordance with the attribute values of the attribute. These are merely introduced as examples and are not necessarily medically correct examples. A doctor or the like may make changes as appropriate to the display positions in accordance with the way of thinking or circumstances of a user of the similar image display apparatus 100.

### Modified Example 1

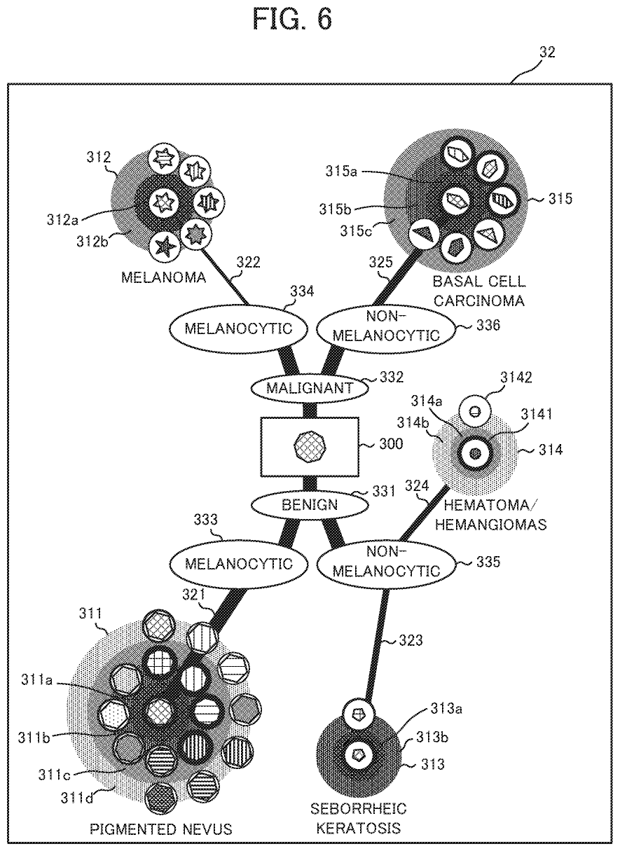

In aforementioned Embodiment 1, in the similar image display processing, displaying is performed as indicated in FIG. 3. Modified Example 1 makes it even easier to understand the similarity relationship and this is described with reference to FIG. 6.

In the similar image display apparatus 100 of Modified Example 1, the image display controller 15 performs processing as follows in step S104 of the similar image display processing (FIG. 4). (The sizes of the circles drawn in the regions for the categories, similar to that in aforementioned Embodiment 1, are larger, the greater the number is of similar images that are classified into the particular category. For example, as illustrated in FIG. 6, a category circle 311 for pigmented nevus is larger than a category circle 312 for melanoma.)
The background of the circles drawn in the regions for the categories is drawn such that background is dark in center and lighter towards the periphery of the circle. For example, as illustrated in FIG. 6, concentric circular graphical shapes 311a, 311b, 311c, and 311d are displayed from the center to the periphery on the background of the circle that is drawn in the region of the category of category circle 311 for pigmented nevus. In FIG. 6, although the darkness changes over two to four levels depending on the size of the circle for each category, the darkness may be set to change smoothly (in gradations) without the use of levels.
The query image is connected, by connection lines, to the image in the center of each individual category.
Regarding the width of the connection lines, the greater the degree of similarity is between a similar image (similar image that is most similar to the query image for a particular category) that is placed at the center of a particular category and the query image that are connected together by a line, the greater the width is of that particular line. For example, the width of a connection line 321 to the category circle 311 for pigmented nevus is thicker than the width of a connection line 322 to the category circle 312 for melanoma.
Also the thickness of the connection line does not need to be displayed at a prescribed thickness that is in accordance with the degree of similarity between the image at the center of the circle and the query image. The thickness of the connection line may be displayed at a prescribed thickness in accordance with a degree of similarity between a prescribed image and the query image. Here, the prescribed image is, for example, an image in a particular category having an n-th (n being an integer that is no less than 1 and no greater than the number of similar images classified in that particular category) greatest degree of similarity with the query image, a lowest degree of similarity, or a middlemost image when arranged in order of degree of similarity. Also, displaying at the prescribed thickness means, for example, that the thickness is displayed thicker the greater the degree of similarity, and displayed thinner the lower the degree of similarity.
At some point along the connections lines 321, 322, 323, 324, 325, the position determiner 13 displays attribute information that is used for determining the individual positions of the categories. For example, in the category for melanoma, a malignant 322 and a melanocytic 334 are displayed as attribute information.
The regions indicating the individual categories are connected together as leaf nodes, by the aforementioned connection lines 321, 322, 323, 324, 325, via a tree structure including the query image as a root node. In other words, the similar images or the like are displayed by a tree structure including the query node as the root node, the regions indicating the individual categories as the leaf nodes, and connection lines, based on attributes of categories corresponding to regions indicating the individual categories, connecting the root node together with the leaf nodes. Also, attribute names (benign 331, malignant 332, melanocytic 333 and 334, non-melanocytic 335 and 336, and the like) indicating attribute information of particular categories are displayed as internal nodes of the connections lines 321, 322, 323, 324, 325 connecting the individual categories together.
Attribute information is largely displayed especially when drawing attention to the information is preferred. (For example, in a case in which similar images are displayed as targets of image data of skin diseases and there are more malignant similar images than benign similar images, the malignant 332 among the attribute information is largely displayed. In FIG. 6, since the benign images are greater in number, the malignant 332 is displayed as the same size as the benign 331.)
Although the individual similar images are displayed as being surrounded by a small circle, the width of the line of the small circle gets thicker, the greater the degree of similarity between the particular similar image and the query image. For example, the width of the line of a small circle 3141 that surrounds a similar image placed at the center of a category circle 314 for hematoma/hemangiomas is thicker than the width of the line of a small circle 3142 that surrounds a similar image placed on the periphery of the category circle 314 for hematoma/hemangiomas.

In step S104 of the similar image display processing (FIG. 4), the displaying of similar images is performed as in, for example, FIG. 6, by the performing of processing as indicated above by the image display controller 15. The following benefits can be obtained by performing the displaying in such a manner.
By placing the individual categories circles 311, 312, 313, 314, 315 in an n-th dimensional space in accordance with the n-types of attributes, the user can intuitively grasp the characteristics of the query image.
By displaying the individual category circles 311, 312, 313, 314, 315 larger in size, the greater the number is of similar images (number of search hits) that are classified into a particular category, the user can intuitively grasp the number of search hits.
By displaying the connections lines 321, 322, 323, 324, 325 connected to the individual categories more thickly, the greater the degree of similarity between the similar image (similar image that is most similar to the query image for a particular category) placed at the center of a particular category with the query image is, the user can intuitively grasp the placement location of the similar image that is most similar to the query image.
By drawing the background of the category circle that is to be drawn in the regions for the individual categories as darkly shaded in center and more lightly shaded towards the periphery of the category circle, the user can visually recognize that the level of severity of the disease examples at the center portion of the circle is high.

### Modified Example 2

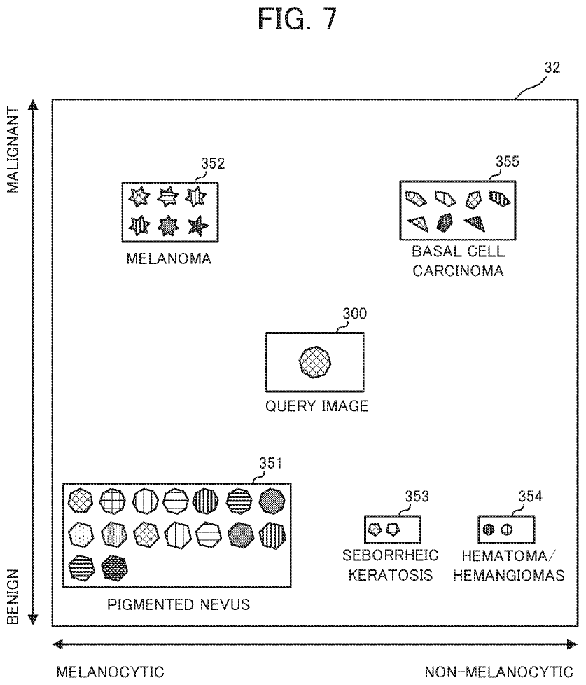

In aforementioned Embodiment 1, although the image display controller 15 collectively places the similar image search results into the individual categories in a concentric circular manner, this is not limiting. Alternative examples of placement include: radially, elliptically shaped, square shaped, and the like. For example, when square-shaped placement is employed, as illustrated in FIG. 7, a more compact displaying can be achieved, and thus, even in a case in which there are many search results, all of the similar images can be displayed at once in a manner that is easily browsable. In the example of FIG. 7, the sizes of squares 351, 352, 353, 354, 355 for the individual categories are made larger by the image display controller 11, the greater the number is of similar images that are classified into a particular individual category. Also, in the similar images are arranged and displayed by the image display controller 15 in descending order of degree of similarity with the query image 300 starting from the upper left corner in each square and if the images extend beyond the right edge, those images are arranged and displayed on the next row below starting back from the left edge.

In aforementioned Embodiment 1, an example is described in which the number of attributes used by the position determiner 13 for determining the positions is two types, these two attributes correspond with two axes (X-axis and Y-axis) of a two-dimensional space, and the position determiner 13 determines the coordinates in the two-dimensional space of the display positions for the similar image search results. However, this example is not limiting. For example, the number of attributes used for determining the positions may be one type and the individual categories may be placed on a linear line (one-dimensional space). In such a case, although the categories are placed on a linear line, since the similar images within the category are placed in a concentric circular manner, placement is ultimately performed in a two-dimensional space.

Alternatively, the number of attributes used for determining the positions may be three types and the individual categories may be placed in a three-dimensional space. In such a case, although the categories and similar images are placed in a three-dimensional space, since the outputter 32 outputs these as a projection onto a two-dimensional space, these can be displayed on a conventional display. Also, in a case in which n of the n-types of attributes used for determining positions is greater than or equal to four, the individual categories may be placed in a virtual n-dimensional space, and ultimately projected onto a two-dimensional space. The types of attributes are not limited to the aforementioned “benign/malignant” and “melanocytic/non-melanocytic” and may also include: “endothelial/non-endothelial”, “metastatic/non-metastatic”, “ductal/non-ductal”, “viral/non-viral”, “size (diameter of a circumscribed ellipses of the disease-affected area, for example)”, “ellipticity (ellipticity of the circumscribed ellipses of the disease-affected area, for example)”, “lesion surface area (surface area of the disease-affected area)”, “contour length (contour length of the outer portion of the disease-affected area)”, “depth of tumor (determined by color (black if shallow, and shifting from brown to gray and finally to pale steel color as the tumor depth deepens)”, “color of disease-affected area (arranged on a color-based axis corresponding to depth of tumor”, “use of a value of a shape (for example, a moment (obtained by performing a moment calculation for a coordinates value of a lesion region, a coordinates value of a contour of lesion region, pixel value of a lesion region, and the like)”, “time (for example, through prolonged observation of size, a time variation of measurement values of size, can be viewed, by taking measurement values of size where time is placed on the horizontal axis and size is placed on the vertical axis)”, and the like.

Also, in aforementioned Embodiment 1, although a case is described using skin diseases as an example, the present disclosure is not limited to the field of dermatology. The present disclosure can be widely applied to fields involving the display of similar images. For example, the present disclosure can also be applied to similar searching of images of flowers, similar searching of microscope pictures of bacteria, and the like.

Also, in aforementioned Embodiment 1, although the similar image display processing is performed by the controller 10, the controller 10 may receive, via the communicator 33, a result processed using an external server, and output the received result to the outputter 32.

Also, the aforementioned Embodiment 1 and Modified Examples 1 and 2 may be combined together as appropriate. For example, by combining Modified Example 1 and Modified Example 2 together, the similar images can be displayed as square shapes for each category and the drawing of connection lines and backgrounds of the square shapes can be performed. As such, the benefits of both Modified Example 1 and Modified Example 2 can be attained. For example, in such a case, the width of the connection line to the similar image (similar image that is most similar to the query image for a particular category) in the upper left corner in the square of the individual categories can be thickened in accordance with the degree of similarity between the particular image and the query image, and the background of a particular square shape can be drawn as darkly shaded in the upper left corner and as more lightly shaded to the lower right.

### Embodiment 2

A display control apparatus 101 according to Embodiment 2 of the present disclosure associates each attribute (“benign/malignant” and “melanocytic/non-melanocytic”, for example) of a disease of a diagnosis target area that is shown in a query image with a particular coordinate axis of the coordinate axes, and displays an index representing the possibility that a disease relates to each attribute as a plot in a space having a number of dimensions equal to the number of attributes of a disease. By displaying in this manner, the display control apparatus 101 makes it easy to grasp the attribute information of a disease of a diagnosis target area. In Embodiment 2, although an example is given in which the disease of the diagnosis target area is a skin disease of a person, as the diagnosis target area (disease), there are many other different types of areas (diseases) that can be diagnosed based on captured images, including the uterus of a person (cervical cancer), the oral cavity of a person (oral cancer), skin (skin cancer) of an animal (cat) and oral cavity of an animal (oral cancer), and the like.

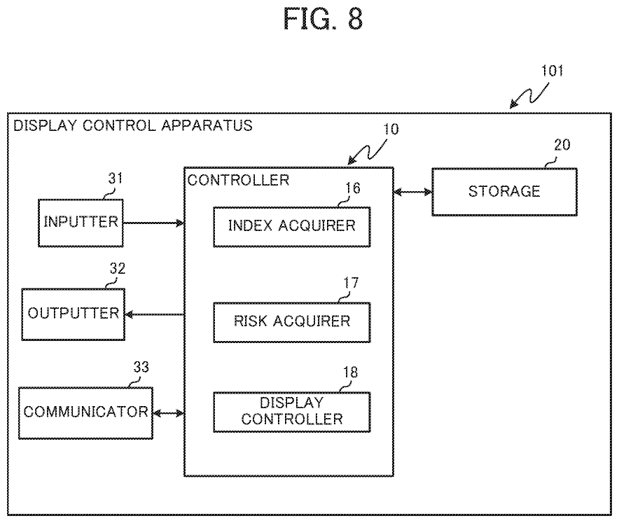

The display control apparatus 101 according to Embodiment 2, as illustrated in FIG. 8, includes the controller 10, the storage 20, the inputter 31, the outputter 32, and the communicator 33.

The controller 10 includes, for example, a CPU, and executes programs stored in the storage 20 to achieve the functions of individual components (index acquirer 16, risk acquirer 17, and display controller 18), which are described further below.

The storage 20 includes the ROM, the RAM, and the like, and stores programs to be executed by the CPU of the controller 10 and necessary data.

The inputter 31 is a device used by a user of the similar image display apparatus 101 to input instructions directed at the similar image display apparatus 101 and input query images. Examples of the inputter 31 include a keyboard, a mouse, a touch panel, a camera, and the like. The controller 10 acquires instructions and query images from the user via the inputter 31. Any device can be used as the inputter 31 as long as the controller 10 can acquire instructions or query images from the user. Moreover, the controller 10 may acquire query images via the communicator 33. The term query image refers to image data of images taken of a diagnosis target area by use of a dermatoscope, for example. The display control apparatus 101 presents, in a manner that is easy to understand by the user, attribute information of a disease of the diagnosis target area that is shown in the query image.

The outputter 32 is a device (a display, interface for the display, or the like) used by the controller 10 to present attribute information of a disease to the user in an easy to understand manner. The display control apparatus 101 may include the outputter 32 as a display, and may display the attribute information or the like on an external display connected via the outputter 32.

The communicator 33 is a device (network interface, for example) for transmitting and receiving data to and from another external device (server storing a database of image data or an image identification device). The controller 10 can acquire image identification results and the like by the image identification device via the communicator 33.

Next, the function of the controller 10 is described. The controller 10 achieves the functions of an index acquirer 16, a risk acquirer 17, and a display controller 18).

The index acquirer 16 uses an identifier to obtain a probability (possibility) of a disease of a diagnosis target area shown in a query image being related to a particular attribute of attributes, and acquires the obtained probability as an index of the particular attribute. This identifier includes, for example, a convolutional neural network, and is trained by use of prescribed image data that is for training in advance. The index acquirer 16 may include such kind of an identifier that is already trained and may cause an external image identification device that includes an image identifier that is already trained to identify a query image, via the communicator 33, and then, the index acquirer 16 may acquire a probability (possibility) of a disease of a diagnosis target area relating to a particular attribute of attributes attained from the identification result as the index of the particular attribute. The index acquired by the index acquirer 16 is not limited to probability. The index acquirer 16 may acquire a more conventional score (conceivably, a score (not necessarily equal to the probability value) being greater in value the greater the possibility, or conversely, a score being greater in value the lower the possibility) as the index.

Here, it is assumed that the index acquirer 16 includes a disease identifier that outputs individual probabilities (hereinafter referred to as “disease-applicable probabilities”) of the disease of the diagnosis target area shown in the query image being one of four particular diseases (melanoma, basal cell carcinoma, pigmented nevus, and seborrheic keratosis). Also, the disease-applicable probabilities obtained by inputting the query image into this disease identifier are assumed to be, for example, 89.0% for melanoma, 4.4% for basal cell carcinoma, 6.4% for pigmented nevus, and 0.2% for seborrheic keratosis. The attributes of these diseases are: “benign/non-melanocytic” for pigmented nevus, “malignant/melanocytic” for melanoma, “benign/non-melanocytic” for seborrheic keratosis, and “malignant/non-melanocytic” for basal cell carcinoma.

In this example, the probability of the attribute of the disease of the diagnosis target area being “malignant” is calculated as 89.0%+4.4%=93.4% and the probability of the attribute of the disease of the diagnosis target area being “benign” is calculated as 6.4%+0.2%=6.6%. Also, the probability of the attribute of the disease of the diagnosis target area being “melanocytic” is calculated as 89.0%+6.4%=95.4% and the probability of the attribute of the disease of the diagnosis target area being “non-melanocytic” is calculated as 4.4%+0.2%=4.6%. The index acquirer 16 acquires the individual probabilities of the attribute of the disease of the diagnosis target area being one of the particular attributes calculated in the aforementioned manner, as indexes representing the individual possibilities of the attribute of the disease of the target area being one of the particular attributes. In particular, the probability of the attribute of the disease of the diagnosis target area being “malignant” and the probability of the attribute of the disease of the diagnosis target area being “benign” are also respectively referred to as the malignant index and the benign index. Likewise, the probability of the attribute of the disease of the diagnosis target area being a prescribed disease attribute such as “melanocytic” or “non-melanocytic” is also referred to as the disease attribute index. Also, in a case in which multiple disease attributes are to be referred to in a distinguishable manner, first, second, and the like are appended to the attribute. For example, among the attributes of the disease of the diagnosis target area, “melanocytic” is the first disease attribute and “non-melanocytic” is the second disease attribute, the probability of “melanocytic” attribute of the disease of the diagnosis target area is referred to as the first disease attribute index, whereas probability of the attribute “non-melanocytic” of the disease of the diagnosis target area is referred to as the second disease attribute index.

The index acquirer 16 does not necessarily use the disease identifier that acquires the disease-applicable probabilities of the diagnosis target area. The index acquirer 16, for example, in place of the disease identifier, alternatively may use an identifier that outputs the probability (malignant index) of the disease of the diagnosis target area being “malignant” or may use an identifier that outputs the probability (disease attribute index) of the attribute of the disease of the diagnosis target area being a prescribed disease attribute such as “melanocytic”.

The risk acquirer 17 acquires a risk index indicating whether or not the risk of the disease is high in a case in which the attribute of the disease is malignant and the attribute of the disease is a prescribed disease attribute. Here, although it is conceivable that there are, as risks, overlook risks (risk of erroneous determination being (no malignant detection) made by the identifier or prognostic risks (neglected risks), the risk acquirer 17 may distinguish between these risks and handle them as separate risk indexes or may handle these values comprehensively as a single risk index. For example, the controller 10 obtains a risk index of overlook risk by, for example, using image data (trial disease case data) other than the training data used for training the disease identifier and/or obtains a risk index of prognostic risk by, for example, using data regarding prognostic risk from a specialist and the like and stores in advance the risk indexes into the storage 20. Also, a risk index may be obtained in advance by using, for example, an external server. The acquirer 17 acquires the risk index obtained in advance by the controller 10 or the external server, for example. In the present embodiment, this risk index is an index indicating the extent of overlook risk of the particular disease when the attribute of the disease is malignant, based on the malignant index of the particular disease, and is pre-generated by risk boundary line generation processing which is described further below.

For example, identifying a melanocytic malignant disease is more difficult than identifying a non-melanocytic malignant disease, and thus the overlook risk for the melanocytic malignant disease is greater, even though the probability of “malignant” (malignant index) is the same for both. In the present embodiment, since a malignant index that is greater than the risk index indicates that overlook risk is high, the risk index for the disease attribute “melanocytic” is lower in value than the risk index for the disease attribute “non-melanocytic”. Therefore, when the disease attribute is “melanocytic”, a risk index of a value that is lower than that of when the disease attribute is “non-melanocytic” is acquired by the risk acquirer 17.

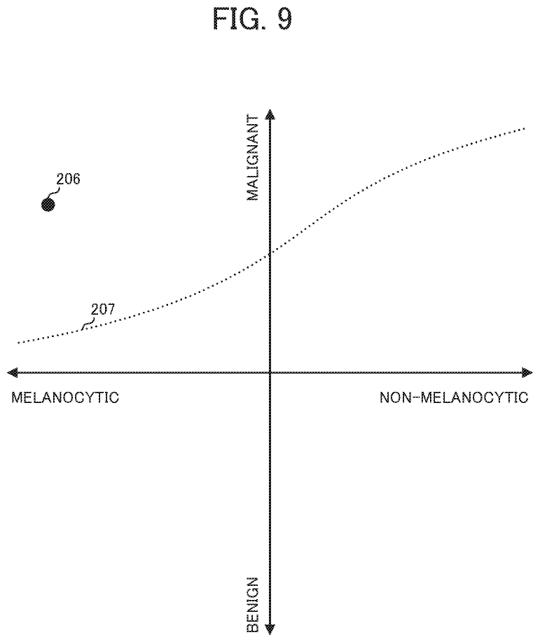

Through the display control processing which is described further below, the display controller 18 causes the display to display multiple indexes, which are acquired by the index acquirer 16, in association with each other. For example, regarding the diagnosis target area shown in the query image, when the index acquirer 16 acquires 93.4% as the index of “malignant” and acquires the 95.4% as the index of “melanocytic”, the display controller 18 causes the displays to display a point 206 as the score corresponding to (95.4% and 93.4%) as illustrated in FIG. 9.

In FIG. 9, the names of the attributes are placed on both ends of both axes with malignant and benign being on the vertical axis and melanocytic and non-melanocytic being on the horizontal axis. However, in actuality, since both axes are based on a single index (the attributes on both ends of both axes are opposite in meaning, for example, malignant means 100% whereas benign means 0%, for example, a single name such as malignant may be placed by itself on the vertical axis and single name such as melanocytic may be placed by itself on the horizontal axis. In FIG. 9, at the point of intersection of the vertical axis with the horizontal axis indicates is where the index of both malignant and benign is 50% and the index of both melanocytic and non-melanocytic is 50%.

Also, the display controller 18 causes the risk index acquired by the risk acquirer 17 and the indexes acquired by the index acquirer 16 to be displayed in association with one another on the display. As an example of this display, the display controller 18 displays, as a risk boundary line 207 indicated by the dotted line in FIG. 9, a risk boundary line generated by the risk boundary line generation processing which is described further below. In FIG. 9, although the point 206 is greater than the risk boundary line 207, this indicates that the risk of the disease of the diagnosis target area shown in the query image is high. Although FIG. 9 illustrates an example where the risk boundary line 207 is based on overlook risk, in a case in which the risk acquire 17 acquires prognostic risk in addition to overlook risk, the display controller 18 may display a risk boundary line that is based on prognostic risk (not illustrated) in addition to the risk boundary line 207 that is based on overlook risk. Also, in a case in which only the prognostic risk is acquired by the risk acquirer 17, the display controller 18 may display boundary line that is based on prognostic risk (not illustrated) by itself, that is, without displaying the boundary line 207 that is based on overlook risk.

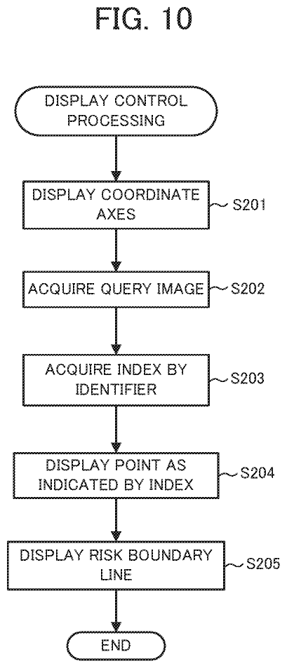

The functional configuration of the display control apparatus 101 is described above. Details of the display control processing performed by the display control apparatus 101 are described with reference to FIG. 10. The display control processing begins when the user instructs the display control apparatus 101, via the inputter 31, to start the display control processing. Also, prior to giving the instruction to begin the display control processing, the user first instructs the display control apparatus 101 regarding the types of attributes that are to be used for the coordinate axes (for example, “benign/malignant” for the vertical axis and “melanocytic/non-melanocytic” for the horizontal axis”.

First, the display controller 18 displays the coordinate axes onto the display (step S201). The coordinate axes that are displayed here are coordinate axes that are based on attributes instructed in advance by the user. For example, in the example illustrated in FIG. 9, the vertical axis is the coordinate axis for malignant (benign/malignant) whereas the horizontal axis is the coordinate axis for melanocytic (melanocytic/non-melanocytic).

Next, the controller 10 of the display controller 101 acquires the query image (step S202). For example, when the user inputs (drags and drops query image into prescribed region of screen of display, for example) the query image into the display control apparatus 101 via the inputter 31, the controller 10 acquires the query image.

Next, the index acquirer 16 inputs the query image into the identifier and acquires the individual attributes (step S203). Step S203 is also referred to as the acquisition step. Then, the display controller 18 displays, on the coordinate axes displayed on the display, the point 206 at the coordinates represented by the index acquired by the index acquirer 16 (step S204). Step S204 is also referred to as the display control step.

Next, the display controller 18 displays, on the display, the risk boundary line 207, stored in the storage 20, having been generated in advance during the risk boundary line generation processing which is described further below (step S205). The display control processing ends upon completion of step S205.

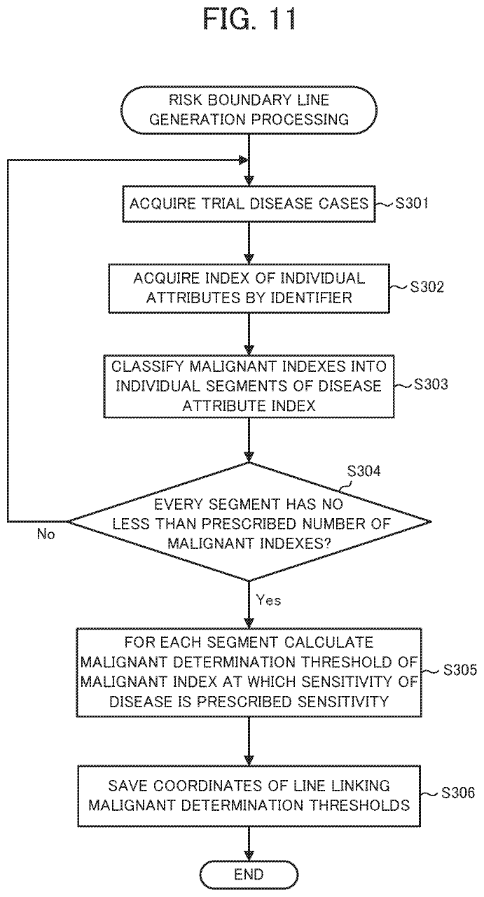

Next, the risk boundary line generation processing is described with reference to FIG. 11. The risk boundary line processing is executed prior to the execution of the display control processing (FIG. 10). Specifically, the risk boundary line processing begins upon the issuance of instructions by the user regarding the attributes that are to be used on the coordinate axes of FIG. 9. However, the risk boundary line generation processing may be executed in advance by an external server or the like. In such a case, the controller 10 acquires the result (risk boundary line coordinates) via the communicator 33 and stores the results into the storage 20. The case in which the risk boundary line generation processing is executed in advance by the controller 10 is described.

First, the controller 10 acquires trial disease case data (not yet used for training the disease identifier) from the storage 20 or via the communicator 33 (step S301). Next, the index acquirer 16 inputs the trial disease case image data into the disease identifier and acquirers the attribute indexes corresponding to the individual coordinate axes (step S302). In the example illustrated in FIG. 9, the attributes are “malignant” and “melanocytic” and here, the index of “malignant” is referred to as the malignant index and the index of “melanocytic” is referred to as the disease attribute index.

Next, regarding the indexes acquired in step S302, the controller 10 classifies the malignant index into the individual segments of the disease attribute index (step S303). Here, for example, if the values of the disease attribute index are from 0% to 100% and the individual segments have a width equal to 10%, then the individual segments of the disease attribute index are ten in number with the disease attribute index values from 0% to below 10% being in segment 1, the disease attribute index values from 10% to below 20% being in segment 2, . . . , and the disease attribute index values from 90% to 100% being in segment 10. For example, if the indexes acquired in step S302 are malignant index 35% and disease attribute index 55%, the controller 10 classifies the malignant index 35% into segment 6.

Next, the controller 10 determines whether or not the malignant indexes classified in step S303 are classified into every segment (all of the segments from segment 1 to segment 10 in the aforementioned example), with every segment having no less than a prescribed number (20, for example) (step S304). If there are any segments with a number of classified malignant indexes less than the prescribed number (NO in step S304), processing returns to step S301 where index classification is repeated using new trial disease case data.

If every segment has a number of classified malignant indexes that is greater than the prescribed number (YES in step S304), the controller 10 calculates, for every segment, a malignant determination threshold of a malignant index at which the sensitivity of the malignant disease is a prescribed sensitivity (95% for example) (in the case where the sensitivity is 95%, for example, a threshold at which 95% are determined as being malignant diseases once a certain number of test disease cases for a malignant disease is identified) (step S305). The lower this threshold is, the easier it is to determine that the attribute of the disease is malignant, and thus sensitivity increases and specificity (accuracy percentage of benign disease cases) decreases.

Then, the controller 10 sets a line, such as a spline curve, linking the malignant determination thresholds of the individual segments together as the risk boundary line and saves the coordinates of the risk boundary line into the storage 20 (step S306). The risk boundary line generation processing ends upon completion of this step. When the points displayed in step S204 of the display control processing (FIG. 10), is above this risk boundary line, it means that the risk of the disease of the diagnosis target area shown in the query image is high.

The aforementioned risk boundary line generation processing merely represents a single example. The following modified examples are also conceivable.
Making modifications in accordance with the disease risk (Prognostic risk goes up since metastability of a melanocytic disease (melanoma and the like) is substantially greater than that of non-melanocytic diseases (basal cell carcinoma and the like) so the risk boundary line is lowered in the region where the probability of the attribute of the disease being melanocytic is high.)
Raising or lowering in accordance with the size of the diagnosis target area (Prognostic risk goes up as the size increases so the risk boundary line is lowered.)
Raising or lower in accordance with the lesion depth of the diagnosis target area (lesion depth is estimated by image processing involving, for example, determination by color of the diagnosis target area and since prognostic risk goes up as the lesion depth increases, the risk boundary line is lowered.)
Raising or lowering in accordance with the size an ulcer as the diagnosis target area or in accordance with the size of the region where bleeding is occurring in the diagnosis target area (Prognostic risk goes up when there is an ulcer and bleeding and goes up even more, the greater the size of the region is in which there is an ulcer and bleeding so the risk boundary line is lowered.)

As described above, the display control apparatus 101, in response to an input query image, can display attribute information of the diagnosis target area shown in the query image in a manner that is easy to understand by use of the coordinates of the point 206 as illustrated in FIG. 9. Also, by also displaying the risk boundary line 207, the extent of the risk of the disease of the diagnosis target area can be grasped through the positional relationship of the point 206 and boundary line 207.

Similar to that in the similar image display apparatus according to Embodiment 1, in the display control apparatus 101 according to Embodiment 2, the following attributes: “endothelial/non-endothelial”, “metastatic/non-metastatic”, “ductal/non-ductal”, “viral/non-viral”, “size of the disease-affected area”, “color of the disease-affected area”, “time (for example, through prolonged observation of size, a time variation of measurement values of size, can be viewed, for example, by taking measurement values of, for example, size where time is placed on the horizontal axis and size is placed on the vertical axis)”, and the like may be used in place of at least one of “benign/malignant” or “melanocytic/non-melanocytic”. Among these attributes, since melanocytic is considered to be the attribute with the highest prognostic risk, in the example illustrated in FIG. 9, an index representing the possibility that the attribute of the disease is melanocytic (melanocytic/non-melanocytic) is placed on the horizontal axis.

In aforementioned Embodiment 2, “benign/malignant” is assigned to the vertical axis and “melanocytic/non-melanocytic” is assigned to the horizontal axis, both as attributes, and the point 206 is displayed on the two-dimensional space. However, the attributes used may be three types and the point 206 may be placed in a three-dimensional space. In such a case, this projection onto a two-dimensional space may be outputted to the outputter 32. Also, in a case in which n of the n-types of attributes used is greater than or equal to four, the point 206 may be placed in a virtual n-dimensional space, and ultimately projected onto a two-dimensional space and outputted to the outputter 32.

### Embodiment 3

A display control apparatus 102 according to Embodiment 3 of the present disclosure displays an attribute of a disease of a diagnosis target area that is shown in a query image together with a probability of the disease of the diagnosis target area being a prescribed disease by using a tree structure including the query image as the root node. By displaying in this manner, the display control apparatus 102 makes it easier to grasp the attribute information of the disease of the diagnosis target area.

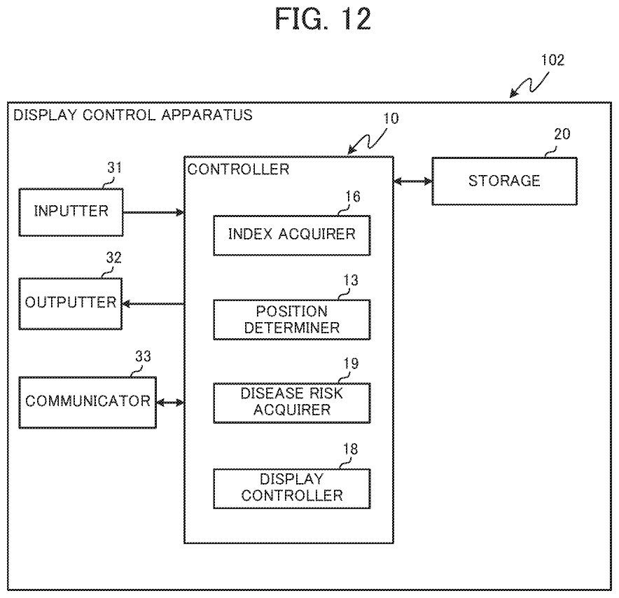

The display control apparatus 102 according to Embodiment 3, as illustrated in FIG. 12, includes the controller 10, the storage 20, the inputter 31, the outputter 32, and the communicator 33. Of these components, the storage 20, the inputter 31, the outputter 32, and the communicator 33 are similar to the storage 20, the inputter 31, the outputter 32, and the communicator 33 that are included in the display control apparatus 101 according to Embodiment 2, and thus descriptions for these similar components are omitted.

The controller 10 includes, for example, a CPU, and executes programs stored in the storage 20 to achieve the functions of individual components (index acquirer 16, position determiner 13, disease risk acquirer 19, and display controller 18), which are described further below.

The index acquirer 16 uses an identifier that identifies a disease among a prescribed number of diseases to obtain a probability (possibility) of a disease of a diagnosis target area shown in a query image being related to a particular attribute of attributes, and acquires the obtained probability as an index of the particular attribute. This identifier includes, for example, a convolutional neural network, and is trained by use of image data that is for training and is prescribed in advance. The index acquirer 16 may include such kind of an identifier that is already trained and may cause an external image identification device that includes an image identifier that is already trained to identify a query image, via the communicator 33, and then the index acquirer 16 may acquire a probability (possibility) of a disease of a diagnosis target area relating to a particular attribute of attributes attained from the identification result as the index of the particular attribute.

Here, the index acquirer 16, as described in the description of the index acquirer 16 according to Embodiment 2, includes a disease identifier that outputs disease-applicable probabilities regarding four diseases (melanoma, basal cell carcinoma, pigmented nevus, and seborrheic keratosis). Also, the disease-applicable probabilities obtained by inputting the query image into this disease identifier are assumed to be, for example, 89.0% for melanoma, 4.4% for basal cell carcinoma, 6.4% for pigmented nevus, and 0.2% for seborrheic keratosis.

In this example, as described in the description of the index acquirer 16 according to Embodiment 2, regarding the indexes representing the individual possibilities of the attribute of the disease of the diagnosis target area being one of the particular attributes, the malignant index is 93.4%, the benign index is 6.6%, the disease attribute index for “melanocytic” is 95.4%, and the disease attribute index for “non-melanocytic” is 4.6%. Also, the index acquirer 16 according to Embodiment 3 also acquires, as the disease indexes, the probabilities that are outputted by the disease identifier. In this example, the disease index for melanoma is 89.0%, the disease index for basal cell carcinoma is 4.4%, the disease index for pigmented nevus is 6.4%, and the disease index for seborrheic keratosis is 0.2%.

The position determiner 13 determines positions where information regarding the individual diseases (categories) for the number of disease indexes that are acquired by the index acquirer 16 is to be displayed, as coordinates in an n-dimensional space based on n-types of attributes (n being an integer greater than or equal to one). More specifically, each attribute of n-types of attributes is associated in one-to-one correspondence with a coordinate axis of n-axes defining the coordinates of the n-dimensional space, and the coordinates indicating the positions where information regarding the individual diseases is to be displayed are determined based on indexes representing the possibilities that the individual diseases relate to a particular attribute corresponding to a particular coordinate axis of the coordinate axes.

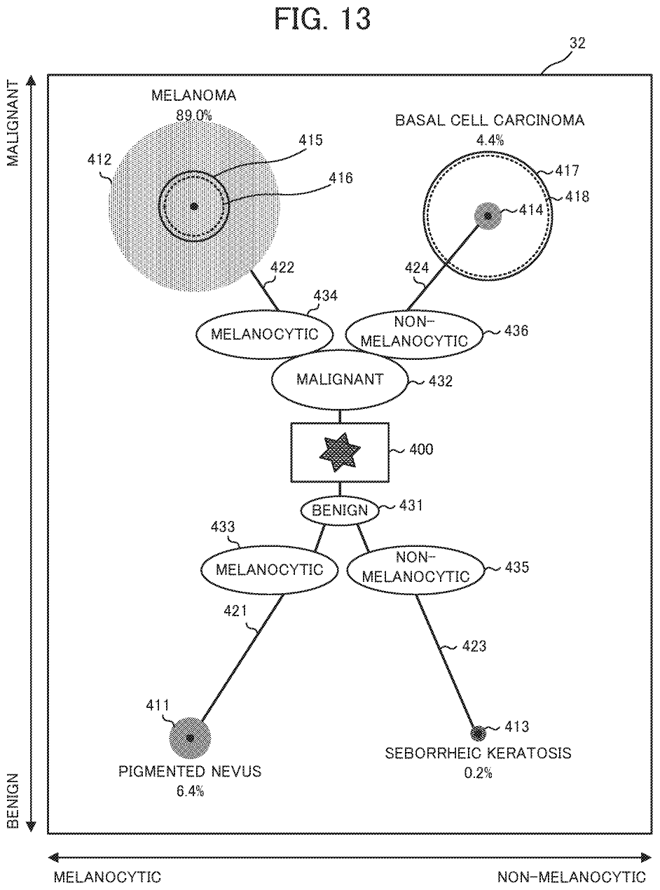

For example, in a case in which the following two types of attributes: “benign/malignant” and “melanocytic/non-melanocytic” are utilized as the aforementioned attributes of n-type of attributes, the position determiner 13 determines the coordinates in a two-dimensional space where the information regarding the individual disease corresponding to the disease indexes acquired by the index acquirer 16 is to be displayed. The position determiner 13, for example as illustrated in FIG. 13, determines the coordinates of the positions where circles (probability circles) representing how high or low the probabilities of the individual diseases of the diagnosis target area in a two-dimensional space are with “benign/malignant” placed on the vertical axis (Y-axis) and “melanocytic/non-melanocytic” placed on the horizontal axis (X-axis). In FIG. 13, benign is placed on the lower side whereas malignant is placed on the upper side on the vertical axis (Y-axis), and melanocytic is placed on the left side whereas non-melanocytic is placed on the right side on the horizontal axis (X-axis).

If, as a specific example, the following four disease names: pigmented nevus, melanoma, seborrheic keratosis, and basal cell carcinoma are considered, the attributes for the diseases are as follows: “benign, melanocytic” for pigmented nevus, “malignant, melanocytic” for melanoma, “benign, non-melanocytic” for seborrheic keratosis, and “malignant, non-melanocytic” for basal cell carcinoma. Therefore, the position determiner 13, as illustrated in FIG. 13, determines the positions for each of the diseases as follows: the lower left region for pigmented nevus, the upper left region for melanoma, the lower right region for seborrheic keratosis, and the upper right region for basal cell carcinoma.

The position determiner 13 may adjust the display positions of the information regarding the diseases as necessary so that the positions where information regarding different diseases is displayed each have different coordinates. Although not displayed in FIG. 13, in a case in which, for example, the index acquirer also acquires a disease index of hematoma/hemangiomas, the attribute of hematoma/hemangiomas, similar to that of seborrheic keratosis, is “benign, non-melanocytic”, so information regarding both diseases will be displayed in the same bottom right region unless the display positions of the information regarding the diseases are adjusted. In such a case, the position determiner 13, for example, may adjust the display positions regarding the individual diseases by shifting the display position of information regarding the seborrheic keratosis to a position that is slightly to the left of the center of the bottom right region and shifting the display position of information regarding hematoma/hemangiomas to a position that is slightly to the right of the center of the bottom right region.

Information of the n-types of attributes that is used for determining the coordinate axes in a space, information of attributes of the respective diseases, and placement information for the individual attributes, for the position determiner 13 to determine the display positions of information regarding the individual diseases, is stored in advance in the storage 20. The position determiner 13 determines the coordinates in the n-dimensional space of positions where information regarding the individual diseases is to be displayed, based on the information of the n-types of attributes, information of the attributes of the respective diseases, and the placement information for the individual attributes that are stored in the storage 20. In the example illustrated in FIG. 13, the information of two types of attributes, namely, the “benign/malignant” attribute and the “melanocytic/non-melanocytic” attribute, are stored in the storage 20 as attribute information. Also, the following information: pigmented nevus is “benign, melanocytic”, melanoma is “malignant, melanocytic”, seborrheic keratosis is “benign, non-melanocytic”, and basal cell carcinoma is “malignant, non-melanocytic” is stored in the storage 20 as the information of the attributes of the respect diseases. Also, the following information: “benign” of the “benign/malignant” attribute is placed on the lower side whereas “malignant” of the “benign/malignant” attribute is placed on the upper side and “melanocytic” of the “melanocytic/non-melanocytic” attribute is placed on the left side whereas “non-melanocytic” of the “melanocytic/non-melanocytic” attribute is placed on the right side is stored in the storage 20 as placement information for the individual attributes.

For each disease, a disease risk acquirer 19 acquires a risk index indicating whether the risk for that particular disease is high or not. Here, although the risk of disease includes prognostic risk (neglected risk in a case when a disease is neglected) or overlook risk (erroneous determination risk where the disease identifier makes a determination that a malignant disease is not a malignant diseases), the disease risk acquirer 19 may distinguish between these risks and handle them as separate risk indexes or may handle these values comprehensively as a single risk index. For example, for melanoma there is greater prognostic risk and overlook risk than that of basal cell carcinoma. Therefore, the disease risk acquirer 19 may acquire, for example, 10% as a risk index of melanoma and 80% as a risk index of basal cell carcinoma. This is an example in which if the disease of the diagnosis target area is melanoma, the risk is high even though the probability (disease index) is 10%, whereas if the disease of the diagnosis target area is basal cell carcinoma, unless the probability (disease index) is greater than or equal to 80%, the risk is not regarded as high, for example. The values of the risk indexes for these individual diseases may be values that are set in advance by a doctor or the like on a per-disease basis. Similar to the processing in the risk boundary line generation processing (FIG. 11) of Embodiment 2, trial disease case data that is different from data used for training may be used and a disease index at which the sensitivity of individual diseases is a prescribed value (95% or 90%, for example) may be obtained in advance as a determination threshold, and the obtained threshold value may be acquired as the risk index.

Through the display control processing which is described further below, the display controller 18 causes the display to display multiple indexes, which are acquired by the index acquirer 16, in association with one another, as a tree structure, as illustrated in FIG. 13. For example, regarding the diagnosis target area shown in the query image, when the index acquirer 16 acquires the following values: 89.0% for melanoma, 4.4% for basal cell carcinoma, 6.4% for pigmented nevus, and 0.2% for seborrheic keratosis as the disease indexes of the individual diseases, the display controller 18, as illustrated in FIG. 13, displays the probability of the disease of the diagnosis target area being pigmented nevus as probability circle 411 that is equivalent in size to 6.4%, displays the probability of the disease of the diagnosis target area being melanoma as a probability circle 412 that is equivalent in size to 89.0%, displays the probability of the disease of the diagnosis target area being seborrheic keratosis as a probability circle 413 that is equivalent in size to 0.2%, and displays the probability of the disease of the diagnosis target area being basal cell carcinoma as a probability circle 414 that is equivalent in size to 4.4%. In FIG. 13, although a dot indicating the center is displayed at the center of the individual probability circles, the displaying of such a dot is optional and the displaying of the dot at the center may be turned on and off by an instruction or the like that is given by the user.

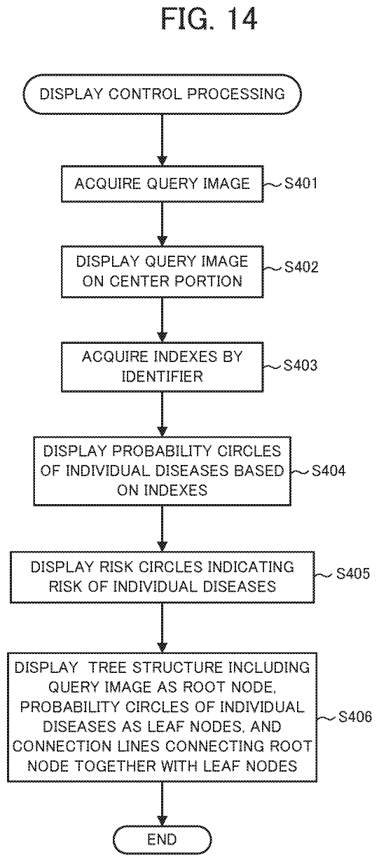

The functional configuration of the display control apparatus 102 is described above. Details of the display control processing performed by the display control apparatus 102 are described next with reference to FIG. 14. The display control processing begins when the user instructs the display control apparatus 102, via the inputter 31, to start the display control processing.

First, the controller 10 of the display control apparatus 102 acquires a query image (step S401). For example, when the user inputs the query image into the display control apparatus 102 via the inputter 31 (drags and drops the query image into a prescribed region on the screen, for example), the controller 10 acquires the query image.

Next, the display controller 18, as illustrated in FIG. 13, displays a query image 400 is displayed on the center portion of the display screen (step S402).

Next, the index acquirer 16 inputs the query image into the disease identifier and acquires the disease indexes of the individual diseases (step S403). Then, the display controller 18, as illustrated in FIG. 13, displays probability circles based on the size of the disease indexes of the individual diseases at the display positions of information regarding the individual diseases whose positions were determined by the position determiner 13 (step S404).

Then, the display controller 18 displays risk circles indicating the size of the risk indexes of the individual disease acquired by the disease risk acquirer 19 are displayed at positions such that the middle of the risk circles coincide with the middle of the corresponding probability circles of the individual diseases (step S405). For example, in FIG. 13, a risk circle 415 that is based on the size of the risk index of melanoma at a sensitivity of 90% is depicted by a solid line and a risk circle 416 that is based on the size of the risk index of melanoma at a sensitivity of 95% is depicted as a dashed line. Also, a risk circle 417 that is based on the size of the risk index of basal cell carcinoma at a sensitivity of 90% is depicted as a solid line and a risk circle 418 that is based on the size of the risk index of basal cell carcinoma at 95% is depicted as a dashed line. Here, “risk index at which sensitivity P % is disease S” is a threshold that is output (probability value of disease S) by the disease identifier in order for disease S to be determined at the sensitivity P % once a certain number of test disease cases for a disease S are identified.

In the example illustrated in FIG. 13, the probability circle 412 for melanoma is larger than the risk circle 415 that is based on the size of the risk index indicating sensitivity 90%, this means that the overlook risk for melanoma is high. Conversely, since the probability circle 414 for basal cell carcinoma is smaller than the risk circle 418 that is based on the size of the risk index indicating sensitivity 95%, this means that the overlook risk for basal cell carcinoma is low.

Next, the display controller 18 displays, as illustrated in FIG. 13, a tree structure including the query image 400 as the root node on the center portion of the display screen, the probability circles 411, 412, 413, 414 of the individual diseases as the leaf nodes, and connection lines 421, 422, 423, 424 connecting the root node together with the leaf nodes (step S406). The display control processing ends upon completion of step S406.

In the displaying of the tree structure in step S406, if the malignant index is larger than the benign index based on the indexes acquired by the index acquirer 16, the display controller 18 displays a malignant node 432 more largely than a benign node 431, as illustrated in FIG. 13. Also, after placing the malignant node 432 and the benign node 431, the individual nodes, namely, melanocytic nodes 433, 434 and non-melanocytic nodes 435, 436 are placed. Then, a tree structure, including, for example, the connections lines 421, 422, 423, 424 respectively extending from these individual nodes to the probability circles 411, 412, 413, 414 of the diseases corresponding to the individual attributes, is displayed.

Although not illustrated in FIG. 13, the display controller 18 may, for example, outline the probability circles of the benign diseases in green and outline the probability circles of the malignant diseases in red in order to make it easy to identify the degree of danger for the individual diseases.

Also, in FIG. 13, although the size of the each probability circle is a size that is in accordance with the size of the probability of the corresponding disease, this is not limiting. Since the extent of the risk differs depending on the disease even when the probability is the same, a large probability circle may be displayed even when the probability of the disease is low in a case where, for example, the risk index acquired by the disease risk acquirer 19 is greater than the probability of that particular disease. Conversely, a small probability circle may be displayed even when the probability of the disease is high in a case where, for example, the risk index acquired by the disease risk acquirer 19 is lower than the probability of that particular disease.

As described above, the display control apparatus 102, in response to the inputted query image, can make it easy to grasp the attribute information of the disease of the diagnosis target area, by indicating the probability of the disease of the diagnosis target area shown in the query image being a prescribed disease by adjusting the sizes of the probability circles, and by displaying the probability circles in a tree structure based on the attributes of the individual diseases, as illustrated in FIG. 13. Also, by displaying the risk circles 415, 416, 417, 418, the extent of the risk of the disease of the diagnosis target area can be grasped through the magnitude relationship between the probability circle 412 and the risk circles 415, 416 and the magnitude relationship between the probability circle 414 and the risk circles 417, 418.

Similar to that in the aforementioned embodiments, in the display control apparatus 102 according to Embodiment 3, the following attributes: “endothelial/non-endothelial”, “metastatic/non-metastatic”, “ductal/non-ductal”, “viral/non-viral”, “size of the disease-affected area”, “color of the disease-affected area”, “time (for example, through prolonged observation of size, a time variation of measurement values of size, can be viewed, for example, by taking measurement values of, for example, size where time is placed on the horizontal axis and size is placed on the vertical axis)”, and the like may be used in place of at least one of “benign/malignant” or “melanocytic/non-melanocytic”. Among these attributes, since melanocytic is considered to be the attribute with the highest prognostic risk, in the example illustrated in FIG. 13, a tree structure is displayed with an index representing the possibility that the attribute of the disease is melanocytic (melanocytic/non-melanocytic) being placed on the horizontal axis, and the index representing the possibility that the attribute of the disease is malignant (benign/malignant) being placed on the vertical axis.

Also, instead of displaying of the malignant node 432, the benign node 431, melanocytic nodes 433, 434, the non-melanocytic nodes 435, 436, and the tree structure including, for example, the connections lines 421, 422, 423, 424 that respectively extend from the these individual nodes to the probability circles 411, 412, 413, 414 of the disease corresponding to the individual attributes, the display controller 18 may alternatively display (i) only the probability circles 411, 412, 413, 414, (ii) only the probability circles 411, 412, 413, 414 together with the risk circles 415, 416, 417, 418, or (iii) only these circles together with a portion of the nodes and connections lines that make up the tree structure.

Also, in aforementioned Embodiment 3, “benign/malignant” is assigned to the vertical axis and “melanocytic/non-melanocytic” is assigned to the horizontal axis, both as attributes, and the tree structure is displayed on a two-dimensional space. However, the attributes used may be three types and the tree structure may be placed in a three-dimensional space. In such a case, this projection onto a two-dimensional space may be outputted to the outputter 32. Also, in a case in which n of the n-types of attributes used is greater than or equal to four, the tree structure may be placed in a virtual n-dimensional space, and ultimately projected onto a two-dimensional space and outputted to the outputter 32.

### Embodiment 4

A display control apparatus 103 according to Embodiment 4 of the present disclosure displays images that are similar to the query image on the periphery of the individual probability circles in addition to displaying the tree structure of the display control apparatus 102 according to Embodiment 3. By performing the displaying in such a manner, the display control apparatus 103 makes it easy to grasp the attribute information of the disease of the diagnosis target area and display the relationship between the similar images in a manner that is easier to understand.

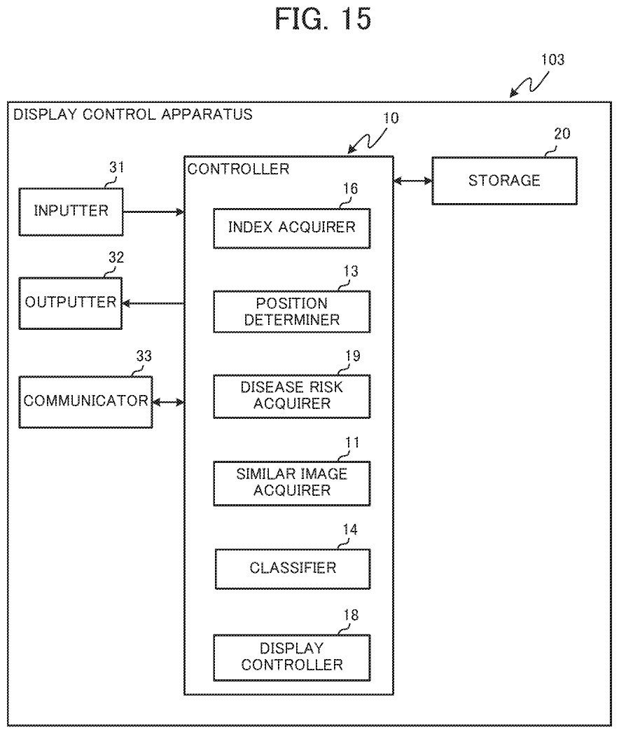

The display control apparatus 103 according to Embodiment 4, as illustrated in FIG. 15, includes the controller 10, the storage 20, the inputter 31, the outputter 32, and the communicator 33. Of these components, the storage 20, the inputter 31, and the outputter 32 are similar to the storage 20, the inputter 31, and the outputter 32 that are included in the display controller apparatus 102 according to Embodiment 3, and thus descriptions for these similar components are omitted. Although the communicator 33 is also similar to the communicator 33 that is included in the display control apparatus 102 according to Embodiment 3, since a similar image searching device or the like, serving as another external device that is a transmission/reception destination for data, is expected, the controller 10 can acquire a similar image search result (images similar to query image) from the similar image searching device via the communicator 33.

The controller 10 includes, for example, a CPU, and executes programs stored in the storage 20 to achieve the functions of individual components (index acquirer 16, position determiner 13, disease risk acquirer 19, similar image acquirer 11, classifier 14, and display controller 18), which are described further below.

The index acquirer 16, the position determiner 13, and the disease risk acquirer 19 are similar to the index acquirer 16, the position determiner 13, and the disease risk acquirer 19 included in the display control apparatus 102 according to Embodiment 3, and thus descriptions for these similar components are omitted.

The similar image acquirer 11, similar to the similar image acquirer according to Embodiment 1, acquires data (image data of similar images and a degree of similarity between the images and the image query) obtained as a result of the similar image search with respect to the query image. Specifically, the similar image acquirer 11 acquires data of images that have a degree of similarity that is greater than or equal to a prescribed threshold in the similar image search and also acquires the degree of similarity. The similar image acquirer 11 may acquire data of similar images obtained as a result of the search by the controller 10 for images that are similar to the query image, and for example, may cause an external similar image searching device to search, via the communicator 33, for images that are similar to the query image, and may also acquire data of the similar images searched by the similar image searching device. Also, the image data is appended with their own corresponding information such as the disease names associated in one-to-one correspondence to the images as tag information.

The classifier 14 classifies image data acquired by the similar image acquirer 11 into a disease identified by a disease identifier that is used by the index acquirer 16. The classifier 14 can classify image data to any disease by use of tag information that is appended to the image data (for example, the disease name is appended as tag information to each image data).

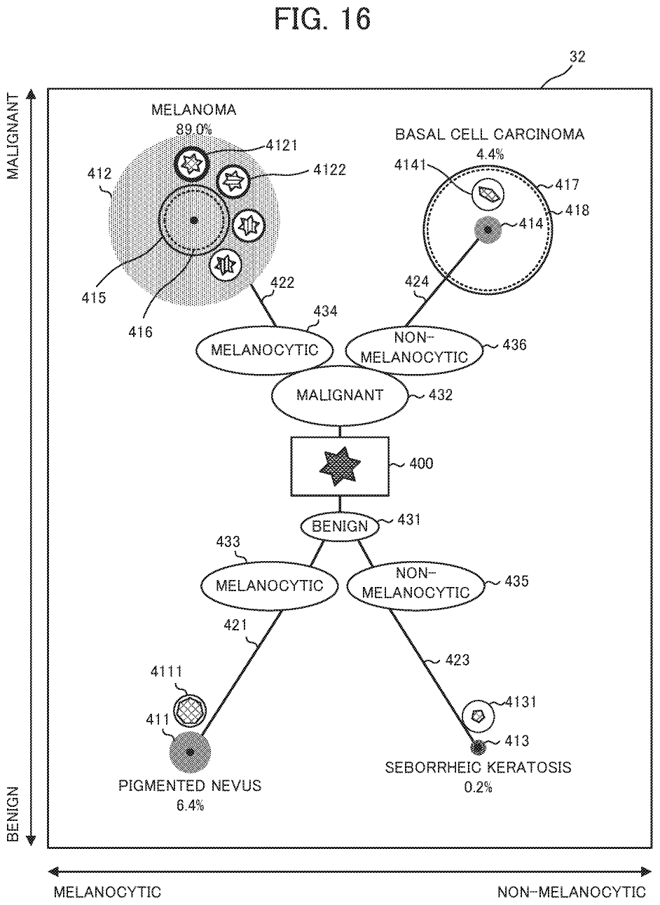

Through the display control processing that is described further below, the display controller 18 performs processing to display data of the similar images acquired by the similar image acquirer 11 on the periphery of the probability circles corresponding to the diseases classified by the classifier 14 as illustrated in FIG. 16, in addition to performing the processing of the display controller 18 according to Embodiment 3.

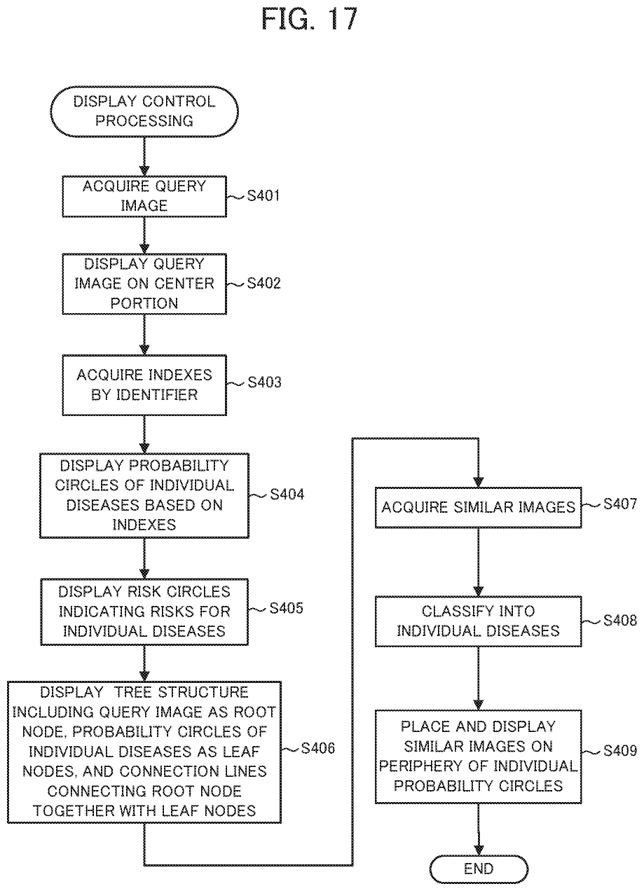

The functional configuration of the display control apparatus 103 is described above. Details of the display control processing performed by the display control apparatus 103 are described next with reference to FIG. 17. The display control processing when the user instructs the display control apparatus 103, via the inputter 31, to start the display control processing. Since the processing in step S401 to step S406 of the display control processing illustrated in FIG. 17 are similar the display control process (FIG. 14) of the display control apparatus 102 according to Embodiment 3, the descriptions for these similar steps are omitted.

When the tree structure is displayed in the processing performed up to step S406, the similar image acquirer 11 next acquires similar images obtained as a result of the similar image search with respect to the query image (step S407). Specifically, similar images that have a degree of similarity with the query image that are greater than or equal with a prescribed threshold are acquired. As such time, the similar image acquirer 11 acquires the similar image together with the degree of similarity the similar image has with the query image.

Then, the classifier 14 classifies, based on the tag information (disease name) appended to the individual similar images, the similar images acquired by the similar image acquirer 11 into the diseases identified by disease identifier that is used by the index acquirer 16 (step S408).

Then, the display controller 18 places and displays the similar images acquired in step S407 by the similar image acquirer 11 on the periphery of the probability circles (or within the probability circles), corresponding to the disease classified in step S408 by the classifier 14 (step S409), on the display. The display control processing ends upon completion of step S409.

Regarding the displaying of the similar images by the display controller 18 in step S409, as illustrated in FIG. 16, placement and displaying is performed in a concentric circular manner on the periphery of the probability circles (or within the probability circles) of the individual disease such that the greater the degree of similarity an image has with the query image, the closer toward the center of the particular probability circle the image is placed. In the example of FIG. 16, the similar image having the greatest degree of similarity with the query image, among the similar images classified into a particular category is placed above the center of the probability circle. Other images are placed clockwise thereafter in descending order of degree of similarity in a concentric circular manner.

Also, although the individual similar images are displayed as being surrounded by a small circle, the width of the line of the small circle gets, thicker the greater the degree of similarity between the particular similar image and the query image. For example, in the example illustrated in FIG. 16, the width of the line of a small circle 4121 that surrounds a similar image placed above the center of the probability circle 412 for melanoma is thicker than the width of the line of a small circle 4122 that surrounds the similar image placed adjacent to the image placed above the center. Furthermore, the width of the line of the small circle 4121 that surrounds the similar image placed above the center of the probability circle 412 for melanoma is displayed more thickly than (i) the thickness of the line of a small circle 4111 that surrounds a similar image that is placed above the probability circle 411 for pigmented nevus, (ii) the thickness of the line of a small circle 4131 that surrounds a similar image that is placed above the probability circle 413 for seborrheic keratosis, and (iii) the thickness of the line of a small circle 4141 that surrounds a similar image placed above the probability circle 414 for basal cell carcinoma. This means that the image having the greatest degree of similarity with the query image among the similar images acquired by the similar image acquirer 11 is the melanoma image (the image surrounded by the small circle 4121 that surrounds the similar image).

As described above, the display control apparatus 103, in response to the inputted query image, can make it easy to grasp the attribute information of the disease of the diagnosis target area, by indicating the probability of the disease of the diagnosis target area shown in the query image being a prescribed disease by adjusting the sizes of the probability circles, and by displaying the probability circles using a tree structure based on the attributes of the individual diseases, as illustrated in FIG. 16. Also, by displaying the risk circles 415, 416, 417, 418, the extend of the risk of the disease of the diagnosis target area can be grasped through the magnitude relationship between the probability circle 412 and the risk circles 415, 416 and the magnitude relationship between the probability circle 414 and the risk circles 417 and 418. Furthermore, since the similar images can be placed and displayed on the periphery of the individual circles (or within the probability circles) in descending order of degree of similarity with the query image, the relationship between similar images can be displayed in a manner that is easier to understand.

Similar to that in the display control apparatus 102 according to Embodiment 3, in the display control apparatus 102 according to Embodiment 4, various attributes can be used. Also, the display controller 18 may alternatively display only a portion of the probability circles 411, 412, 413, 414, the risk circles 415, 416, 417, 418, the query image 400, similar images, the nodes and connections lines that make up the tree structure. Also, n of the n-types of attributes for the tree structure is not limited to two (tree structure in a two-dimensional space). The tree structure may be placed on an n-dimensional space and then ultimately outputted to the outputter 32 as a projection on a two-dimensional space.

Also, although the aforementioned Embodiments 2, 3, and 4 use skin disease as an example, the present disclosure is not limited to the field of dermatology. The present disclosure can be widely applied to fields involving the identification of images with use of an identifier. For example, the present disclosure can also be applied to the identification of types of flowers by using images of flowers, and the identification of bacteria by using microscope pictures of bacteria. Also, any approach may be used to achieve these identifiers. For example, a deep neural network (DNN) such as a convolutional neural network (CNN) may be used to achieve these identifiers or alternatively a support vector machine (SVM), logistic regression, or the like may be used to achieve the identifiers.

Also, although the controller 10 performed the display control processing in the aforementioned Embodiments 2, 3, and 4, the controller 10 may receive, via the communicator 33, a result from causing an external server to perform processing equivalent to the display control processing and output the result to the outputter 32.

Also, the aforementioned embodiments and modified examples may be combined together as appropriate. Although Embodiment 4 can be regarded as an embodiment in which a portion of Embodiment 1 is combined together with Embodiment 3, conversely, a portion of Embodiment 3 may be combined together with Embodiment 1. In such a case, the individual category circles illustrated in FIG. 3 may be substituted with the probability circles each indicating the size of the probability of a disease corresponding to an individual category and the values of the probabilities of the diseases corresponding to the individual categories and the risk circles may also be displayed. In doing so, the similar images can be referenced while visually confirming the probabilities of the individual diseases and the risks, thereby improving usefulness during diagnosis. Also, the shape of the probability circles and risk circles in Embodiment 3 and Embodiment 4 are not limited to circles. Other appropriate shapes (for example, n-sided shapes including triangles, squares, and the like and symbol shapes including hearts, stars, and the like) may be used.

The individual functions of the similar image display apparatus 100 and the display control apparatuses 101, 102, 103 may also be executed by a computer such an ordinary personal computer (PC). Specifically, in the aforementioned embodiments, the program for the similar image display processing that is performed by the similar image display apparatus 100 and the program for the display control processing that is performed by the display control apparatuses 101, 102, 103 are described as being stored in advance in the ROM of the storage 20. However, the program may be stored in, and distributed through, a non-transitory computer-readable recording medium such as a flexible disk, a compact disc read-only memory (CD-ROM), a digital versatile disc (DVD), a magneto-optical disc (MO), a memory stick, or a universal serial bus (USB), and may be installed into a computer to enable the computer to achieve the above-described individual functions.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

What is claimed is:

1. A similar image display control apparatus comprising:
a processor configured to
acquire similar images obtained as a result of a similar image search with respect to a query image,
set categories into which the acquired similar images are to be classified,
determine, in a space having a prescribed number of dimensions that is no less than two, coordinates indicating a position of each category region in accordance with attributes of types equal in number to the number of dimensions, the category region being a region indicating one of the set categories,
classify the acquired similar images into the set categories, and
place the similar images, classified into each of the categories, within the category region positioned in a position as indicated by the determined coordinates and cause a display to display the placed similar images.

2. The similar image display control apparatus according to claim 1, wherein the processor places the similar images within the category region based on a degree of similarity with the query image and causes the placed similar images to be displayed on the display.

3. The similar image display control apparatus according to claim 1, wherein the processor displays on the display the category region using a circle larger in size, the greater a number is of similar images that are classified into a category corresponding to the category region.

4. The similar image display control apparatus according to claim 3, wherein the processor causes a circumferential line of a circle, indicating the category region, to be more thickly displayed on the display, the greater the degree of similarity is between the query image and a similar image having the greatest degree of similarity with the query image among the similar images classified into the category corresponding to the category region.

5. The similar image display control apparatus according to claim 3, wherein the processor places the similar images within the category region in a concentric circular manner and causes the concentric circularly-placed similar images to be displayed on the display.

6. The similar image display control apparatus according to claim 5, wherein the processor places the similar images closer to a center of the category region, the greater the degree of similarity these similar images have with the query images, and causes the placed similar images to be displayed on the display.

7. The similar image display control apparatus according to claim 5, wherein the processor causes a concentrically circular graphical shape to be displayed on a background of the similar images placed and displayed in a concentric circular manner on the display.

8. The similar image display control apparatus according to claim 1, wherein the processor associates each attribute with an individual coordinate axis of the space and determines coordinates indicating a position of the category region, the coordinates being in accordance with an attribute value of the attribute of the category of the space.

9. The similar image display control apparatus according to claim 1, wherein the processor connects to the category region a connection line that is based on the attribute of the category corresponding to the category region and causes the connected connection line to be displayed on the display.

10. The similar image display control apparatus according to claim 1, wherein the processor causes the similar images to be displayed on the display by a tree structure including the query image as a root node, the category region indicating the category as a leaf node, and a connection line based on the attribute of the category corresponding to the category region connecting the root node together with the leaf node, and causes an attribute name indicating information of the attribute of the category to be displayed on the display as an internal node of the connection line.

11. The similar image display control apparatus according to claim 9, wherein the processor causes a thickness of the connection line connected to the category region corresponding to the category into which the similar images are classified, to be displayed on the display at a prescribed thickness in accordance with a degree of similarity between a query image and a prescribed similar image among the similar images classified into the category corresponding to the category region.

12. The similar image display control apparatus according to claim 1, wherein the processor causes the query image and one or more than one similar image, selected by a user, among the similar images, to be displayed on the display in an enlarged manner.

13. The similar image display control apparatus according to claim 1, wherein each of the categories is a category of a skin disease name.

14. The similar image display control apparatus according to claim 13, wherein the attributes are two types, one being benign/malignant and the other being melanocytic/non-melanocytic.

15. A similar image display control system comprising:
a similar image display control apparatus; and
a display, wherein
the similar image display control apparatus includes a processor is configured to:
acquire similar images obtained as a result of a similar image search with respect to a query image,
set categories into which the acquired similar images are to be classified,
determine, in a space having a prescribed number of dimensions that is no less than two, coordinates indicating a position of each category region in accordance with attributes of types equal in number to the number of dimensions, the category region being a region indicating one of the set categories,
classify the acquired similar images into the set categories, and
place the similar images classified into each of the categories, within the category region positioned in a position as indicated by the determined coordinates and cause the display to display the placed similar images.

16. A similar image display control method comprising:
acquiring similar images obtained as a result of a similar image search with respect to a query image;
classifying the acquired similar images into categories; and
determining, in a space having a prescribed number of dimensions that is no less than two, coordinates indicating a position of a category region being a region indicating one of the categories, in accordance with attributes of types equal in number to the number of dimensions, placing the similar images, classified into each of the categories, within the category region positioned in position as indicated by the determined coordinates, and causing a display to display the placed similar images.

17. A non-transitory computer-readable recording medium storing a program for causing a computer to execute processing comprising:
acquiring similar images obtained as a result of a similar image search with respect to a query image;
classifying the acquired similar images into categories; and
determining, in a space having a prescribed number of dimensions that is no less than two, coordinates indicating a position of a category region being a region indicating one of the categories, in accordance with attributes of types equal in number to the number of dimensions, placing the similar images, classified into each of the categories, within the category region positioned in a position as indicated by the determined coordinates, and causing a display to display the placed similar images.

18. A display control apparatus comprising:
a processor configured to
acquire a malignant index representing a possibility that an attribute of a disease of a diagnosis target area is malignant and a first disease attribute index representing a possibility that an attribute of the disease of the diagnosis target area is a prescribed first disease attribute, and
cause the acquired malignant index and the acquired first disease attribute index to be displayed in association with each other on a display.

19. The display control apparatus according to claim 18,
wherein the processor acquires a risk index representing whether or not the risk of a disease, when the attribute of the disease is malignant and the attribute of the disease is the first disease attribute, is high, and
causes the acquired risk index to be displayed on the display in association with the acquired malignant index and the acquired first disease attribute index.

20. The display control apparatus according to claim 18, wherein the processor
further acquires a benign index representing a possibility that the attribute of the disease of the diagnosis target area is benign and a second disease attribute index representing a possibility that the attribute of the disease of the diagnosis target area is a second disease attribute that is different from the first disease attribute, and
causes the acquired malignant index, the acquired first disease attribute index, the acquired benign index, and the acquired second disease attribute index to be displayed in association with one another on the display.

21. The display control apparatus according to claim 20, wherein the processor
acquires a risk index representing whether or not the risk of the disease is high, and displays on the display the acquired risk index in association with the acquired malignant index, the acquired first disease attribute index, the acquired benign index, and the acquired second disease attribute index.

22. The display control apparatus according to claim 20, wherein the processor
determines coordinates of a position displaying information regarding a disease,
further acquires a disease index representing a possibility that the disease of the diagnosis target area is a prescribed disease,
causes the acquired indexes to be displayed in association with one another by a tree structure including a query image as a root node, a probability circle whose size is based on the acquired disease positioned in a position as indicated by the determined coordinates as a leaf node, and a connection line, based on the attributes of the disease of the diagnosis target area connecting the root node together with the leaf node.

23. The display control apparatus according to claim 20, wherein the diagnosis target area is skin and the first disease attribute is melanocytic and the second disease attribute is non-melanocytic.

24. The display control apparatus according to claim 18, wherein the processor
outputs probabilities of the disease of the diagnosis target area being related to the individual attributes, and
further acquires the outputted probabilities of the disease of the diagnosis target area being related to the individual attributes, as indexes of the individual attributes.

25. A display control system comprising:
a display control apparatus; and
a display, wherein
the display control apparatus
acquires a malignant index representing a possibility that an attribute of a disease of a diagnosis target area is malignant and a first disease attribute index representing a possibility that an attribute of the disease of the diagnosis target area is a prescribed first disease attribute, and
causes the acquired malignant index and the acquired first disease attribute index to be displayed in association with each other on the display.

26. A display control method comprising:
acquiring a malignant index representing a possibility that an attribute of a disease of a diagnosis target area is malignant and a first disease attribute index representing a possibility that the attribute of the disease of the diagnosis target area is a prescribed first disease attribute; and
causing the acquired malignant index and the acquired first disease attribute index to be displayed in association with each other on a display.

27. A non-transitory computer-readable recording medium storing a program for causing a computer to execute processing comprising:
acquiring a malignant index representing a possibility that an attribute of a disease of a diagnosis target area is malignant and a first disease attribute index representing a possibility that the attribute of the disease of the diagnosis target area is a prescribed first disease attribute; and
causing the acquired malignant index and the acquired first disease attribute index to be displayed in association with each other on a display.
